(19) United States
(12) Patent Application Publication
(10) Pub. No.: US 2026/0131093 A1
(43) Pub. Date: May 14, 2026

(54) PORTABLE VENTILATOR SUITABLE FOR CARDIOPULMONARY RESUSCITATION AND CONTROL METHOD THEREFOR

(71) Applicant: SHANDONG UNIVERSITY, Jinan (CN)

(72) Inventors: Ke LI, Jinan (CN); Bozhi DING, Jinan (CN); Yuguo CHEN, Jinan (CN); Feng XU, Jinan (CN); Jiali WANG, Jinan (CN); Chang PAN, Jinan (CN); Jiaojiao PANG, Jinan (CN); Yuan BIAN, Jinan (CN); Yibin LI, Jinan (CN)

(21) Appl. No.: 19/119,866

(22) PCT Filed: Jun. 26, 2024
(86) PCT No.: PCT/CN2024/101631

§ 371 Date: Apr. 10, 2025

(30) Foreign Application Priority Data
Jun. 20, 2023 (CN) 202310737200.4

(51) Int. Cl. A61M 16/00; A61H 31/00; A61M 16/10; A61M 16/12; A61M 16/20

(52) U.S. Cl. CPC A61M 16/024; A61H 31/005; A61M 16/0003; A61M 16/0057; A61M 16/1005; A61M 16/125; A61M 16/203; A61H 2201/107; A61H 2201/5071; A61H 2201/5087; A61H 2230/425; A61M 2016/0027; A61M 2016/0039; A61M 2016/1025; A61M 2205/3365; A61M 2230/46

(57) ABSTRACT

The present invention provides a portable ventilator suitable for cardiopulmonary resuscitation (CPR) and a control method thereof. According to the present invention, the number of external chest compressions in a CPR process can be automatically recognized on the basis of reverse airflow, and a compression period and a relaxation period can be determined. The portable ventilator has an automatic 30:2 ventilation mode suitable for non-intubated patients and a variable-volume ventilation mode suitable for patients with advanced airways. The present invention can improve the efficiency of first aid and avoid barotrauma, is suitable for the first aid during CPR, and can achieve precise flow control.

### CROSS-REFERENCES TO RELATED APPLICATIONS

The present invention claims priority benefits to Chinese Patent Application number 202310737200.4 entitled “PORTABLE VENTILATOR SUITABLE FOR CARDIOPULMONARY RESUSCITATION AND CONTROL METHOD THEREOF”, filed on 20 Jun. 2023, with the China National Intellectual Property Administration (CNIPA), the entire contents of which are incorporated herein by reference.

### TECHNICAL FIELD

The present invention pertains to the field of ventilator control technologies, and relates to a portable ventilator suitable for cardiopulmonary resuscitation and a control method thereof.

### BACKGROUND

The statements in this section are merely provided as background information related to the present invention and do not necessarily constitute prior art.

Cardiac arrest (CA) refers to a sudden cessation of the heart's pumping function, resulting in the disappearance of arterial pulses and heart sounds, and severe ischemia and hypoxia of vital organs, leading to death. As CA is one of the main causes of death in patients, out-of-hospital cardiac arrest (OHCA) features a lower survival rate.

Cardiopulmonary resuscitation (CPR) is an important method for treating cardiac arrest, generally consisting of defibrillation, cardiopulmonary resuscitation, and ventilatory support. In OHCA, to ensure patients without an advanced airway obtain adequate perfusion, non-invasive ventilation and a “30:2” ventilation mode are used. However, the “30:2” ventilation mode requires a rescuer to count compressions, activating the ventilator or using a rescue bag for 2-time ventilations after every 30-time external chest compressions. After endotracheal intubation and establishment of an advanced airway, the CPR process adopts continuous compressions and continuous ventilation, with a ventilation frequency of 10-time ventilations per minute. The advanced airway mode ensures that the patient can receive sufficient oxygen supply during CPR.

However, the inventors have known that existing ventilators are unable to effectively perform a “30:2” ventilation and advanced airway ventilation, which are mainly manifested in: for the “30:2” ventilation, existing ventilators cannot actively identify an occurrence of a compression event, cannot automatically count the number of compressions, and cannot autonomously perform ventilation tasks, thus the ventilation process requires manual operation; for the advanced airway mode, modern ventilators use modes of pressure support ventilation, volume-controlled ventilation (VCV), or continuous positive pressure ventilation to provide continuous positive pressure ventilation. However, an overlap of an inspiratory period of the positive pressure ventilation with a compression period of the external chest compression (ECC) results in a significant increase in airway pressure of the patient, raising the risk of barotrauma.

### SUMMARY

To resolve the foregoing problem, the present invention provides a portable ventilator suitable for cardiopulmonary resuscitation and a control method thereof, can automatically identify the number of external chest compressions during a CPR process based on a reverse airflow and determine a compression period and a relaxation period, having an automatic “30:2” ventilation mode suitable for non-intubated patients and a variable-VCV mode suitable for patients with advanced airways, improving emergency efficiency and avoiding barotrauma, so as to be more suitable for emergency rescue during CPR with precise flow rate control.

According to some embodiments, the present invention adopts the following technical solution.

A portable ventilator suitable for cardiopulmonary resuscitation, including a fan, an air-oxygen mixing device, a pipeline, a controller, and a flow sensor, wherein:
an inlet of the fan is connected to the air-oxygen mixing device, and an outlet of the fan is connected to a first end of an oxygen sensor;
a second end of the oxygen sensor is connected to an air-inlet of the flow sensor being used to monitor a flow rate, an outlet of the flow sensor is connected to the pipeline, and a control switch is provided on pipeline and is used to close the pipeline;
a pressure sensor is further provided on the pipeline and is used to monitor an air pressure in the pipeline; and
the controller is communicated with the flow sensor and the pressure sensor and is configured to perform a feedback control on the fan based on monitoring data of sensors, ensuring provision of a constant flow rate of air.

In an optional implementation, a first end of the air-oxygen mixing device is connected to the atmosphere and a second end is connected to a proportional solenoid valve, and an inlet of the proportional solenoid valve is connected to an oxygen source.

In an optional implementation, the portable ventilator further includes a power supply module being configured to supply voltage to other electronic devices.

In an optional implementation, the controller is further configured to compare an actual flow rate obtained by the flow sensor with a preset flow rate, and adjust a flow rate of the ventilator based on a difference of the comparison, and generate a PWM (Pulse-Width Modulation) value required by the fan.

In an optional implementation, the controller is further configured to determine whether there is a reverse airflow caused by the compression based on time and flow rate parameters during CPR, count reverse airflows caused by compressions, read a number of the compressions when no advanced airway is established, and carry out ventilations after the compressions.

In an optional implementation, the controller is further configured to determine optimized control parameters after the advanced airway is established, control the flow rate in the airway, determine a compression period and a relaxation period of ECC based on the reverse airflow caused by the compression, and reduce a rotation speed of the fan or turn off the fan during the compression period, or increase the rotation speed of the fan during the relaxation period.

A control method of the ventilator mentioned above, including the following steps:
obtaining monitoring data of a flow sensor and a pressure sensor;
comparing an actual flow rate obtained by the flow sensor with a preset flow rate, and adjusting a flow rate of a ventilator based on a comparison difference to generate a PWM value required by a fan;
during CPR, determining whether there is a reverse airflow caused by the compression based on time and flow rate parameters, counting reverse airflows caused by compressions; when no advanced airway is established, reading a number of the compressions, and carrying out ventilations after the compressions; and
determining optimized control parameters after the advanced airway is established, controlling the flow rate in the airway; determining a compression period and a relaxation period of ECC based on the reverse airflow caused by the compression, and reducing a rotation speed of the fan or turn off the fan during the compression period, or increasing the rotation speed of the fan during the relaxation period.

In an optional implementation, determining whether there is the reverse airflow caused by the compression based on time and flow rate parameters specifically includes:
taking a peak pressure read for the first time as a peak flow value of the first time, after entering the n^{th} compression, obtaining a peak flow value of the n^{th} time, and extracting a rise velocity of the peak flow value of the n^{th} time, wherein n is greater than 1;
when the rise velocity is less than a first threshold, considering that there may be the reverse airflow caused by the compression, otherwise discarding the peak flow value of the n^{th} time;
extracting a time interval Tₙ, determining whether the Tₙ exceeds a second threshold; when the Tₙ is less than the second threshold, determining as an invalid peak flow, and not recording; and
determining whether the peak flow value of the n^{th} time that meets conditions of the rise velocity and the time interval exceeds a third threshold, determining the peak flow value of the n^{th} time that exceeds the third threshold as a valid peak flow, and increasing the compressions by one count.

In an optional implementation, determining optimized control parameters specifically includes: iteratively calculating optimized proportional, integral, and differential coefficients by using an AO-PID (Analog Output-Proportional Integral Derivative) control algorithm.

In an optional implementation, during the CPR, the PWM value is able to be re-adjusted based on the peak flow value of the reverse airflow caused by the compression, to achieve a flow rate adjustment in the compression period and the relaxation period.

In an optional implementation, extracting the peak flow value to determine whether there is a generation of the reverse airflow caused by the compression, locating a zero position of flow rate of the reverse airflow caused by the compression after the reverse airflow caused by the compression has been determined, simultaneously determining whether a flow acceleration at the zero position is greater than 0, wherein if the flow acceleration at the zero position is greater than 0, determining, by the ventilator, that the zero position is an inspiratory period, and if the flow acceleration of the zero position is less than 0, determining that the zero position is an expiratory period.

For the inspiratory period, adjusting the PWM value to PWMᵢ=K₁*PF/Fₛ; and for the expiratory period, adjusting the PWM value to PWMₑ=K₂*PF/Fₛ, where PWM; and PWMₑ are PWM values of the fan at the inspiratory period and the expiratory period respectively, K₁ and K₂ are gain coefficients, PF is the peak flow value of the reverse airflow caused by the compression, and Fₛ is a target flow rate value under normal ventilation conditions, and closing the pipeline when the PWMₑ of the expiratory period is less than a set threshold.

Compared with the prior art, the present invention has the following beneficial effects:

The present invention provides a portable emergency ventilator that automatically identifies the number of chest compressions during CPR based on the reverse airflow and determines the compression period and the relaxation period. The ventilator has a simple structure and a small volume and features automatic “30:2” ventilation for non-intubated patients and the variable-VCV mode for patients with an advanced airway, freeing medical personnel, improving emergency efficiency, and avoiding barotrauma, so as to be more suitable for emergency rescue during CPR. Precise flow control of the ventilator is ensured using a new control method for brushless DC motor.

To make the above objectives, features and advantages of the present invention more comprehensible, preferred embodiments are described as examples below in detail with accompanying drawings.

### BRIEF DESCRIPTION OF THE DRAWINGS

The accompanying drawings of this specification which constitute a part of the present invention provide further comprehension of the present invention. The schematic examples of the present invention and description thereof are intended to explain the present invention and do not constitute an improper limitation to the present invention.

FIG. 1 is a structural diagram of a portable emergency ventilator.

FIG. 2 is a diagram of a control process of identifying the number of compressions.

FIG. 3 is a flowchart of a VCV control of a new portable emergency ventilator.

FIG. 4 is a diagram of a control process of an AO-PID algorithm.

FIG. 5 is a diagram of a control process of variable flow rate control.

In drawings: 1—proportional solenoid valve, 2—power supply, 3—ball valve, 4—pressure sensor, 5—pipeline, 6—flow sensor, 7—oxygen sensor, 8—micro-controller, 9—fan, and 10—air-oxygen mixing device.

### DETAILED DESCRIPTION

The present invention is further described below with reference to the accompanying drawings and specific embodiments.

It should be noted that the following detailed descriptions are illustrative and are intended to further describe the present invention. Unless otherwise indicated, all technical and scientific terms used in this specification have the same meaning as commonly understood by those skilled in the art to which the present invention pertains.

It should be noted that the terms used herein are solely for describing specific implementations and are not intended to limit exemplary implementations of the present invention. As used herein, unless otherwise indicated by context, a singular form is intended to also include a plural form. Additionally, it should be understood that when the term “include” and/or “comprise” is used in this specification, it indicates the presence of features, steps, operations, devices, assemblies, and/or their combinations.

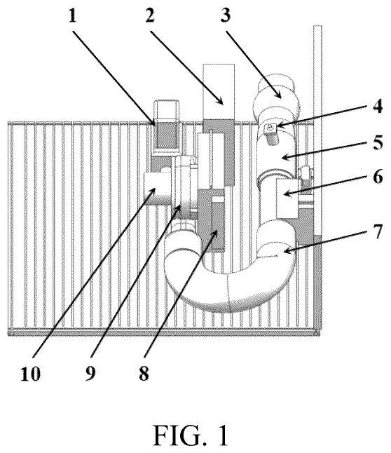

As shown in FIG. 1, a portable ventilator suitable for cardiopulmonary resuscitation mainly includes a fan 9, an oxygen sensor 7, an air-oxygen mixing device 10, a flow sensor 6, a pipeline 5, and a microcontroller 8. The specific connection relationships and functions of the portions are as follows:
an air power source of the ventilator is from the fan 9, and using a PWM signal for regulating a rotation speed of the fan to control a flow rate at the outlet. The outlet of the fan is connected to the oxygen sensor 7, and an air-inlet of the fan 9 is connected to the air-oxygen mixing device 10 for mixing oxygen and air. A first end of the air-oxygen mixing device 10 is connected to the atmosphere, and a second end is connected to the proportional solenoid valve 1. An inlet of the proportional solenoid valve is connected to an oxygen source. A second end of the oxygen sensor 7 is connected to an air-inlet of the flow sensor 6, enabling the flow sensor to monitor a flow rate in an airway in real-time. An outlet of the flow sensor 6 is connected to the pipeline 5.

The pressure sensor 4 is fixed to the pipeline 5 to capture the pressure in the airway in real-time.

The ball valve 3 is connected to a second end of the pipeline 5 to close the ventilator pipeline. The ball valve is added to the ventilator pipeline for closing the ventilator pipeline during the compression period to avoid high-pressure injuries and preventing the fan from stopping, thereby accelerating the switch between inhalation and exhalation.

The microcontroller 8 is used to receive data from the flow sensor 6 and the pressure sensor 4, and to perform feedback control on the fan to ensure the provision of constant flow.

The battery module 2 provides the voltage required by the flow sensor 6 and the fan 9, the voltage required by the pressure sensor 4 and the microcontroller 8, and the voltage required by the automatic ball valve, the pressure sensor, and the proportional solenoid valve.

The voltages of the above means are not necessarily the same. When they are different, the battery module has multiple corresponding output terminals. The battery module can use existing technology or means, which are not elaborated herein.

The reverse airflow (hereinafter referred to as RF) generated by compression is captured for automatically completing a “30:2” ventilation. This portable ventilator uses a flow sensor to capture the flow rate in the airway, determines whether the RF is caused by compression based on time and flow parameters, and counts the RF. In a case where an advanced airway has not been established, the number of compressions is automatically read and ventilation is automatically performed after the compression ends to achieve a “30:2” ventilation mode.

In the specific control process, the RF caused by compression is captured for automatically completing the “30:2” ventilation. This portable ventilator uses a flow sensor to capture the flow rate in the airway, determines whether the RF is caused by compression based on time and flow parameters, and counts the RF. In a case where an advanced airway has not been established, the number of compressions is automatically read and ventilation is automatically performed after the compression ends to achieve the “30:2” ventilation mode.

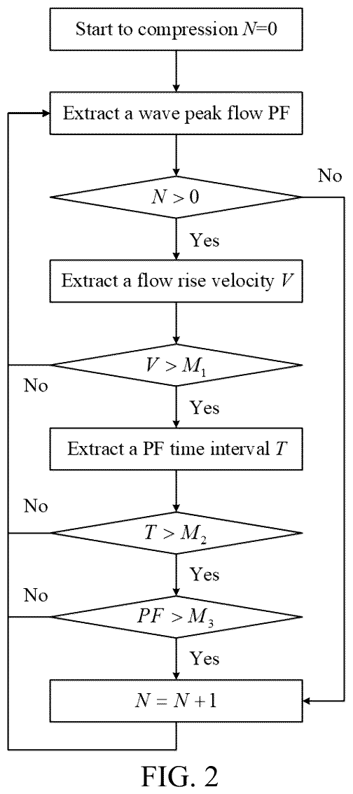

Achieving the identification and counting of RF allows the ventilator to automatically complete the “30:2” ventilation. Because a compression causes a RF, each RF produces not only one peak flow; sometimes there is a secondary wave after a RF. This algorithm eliminates the interference of the secondary wave on the primary wave based on the peak flow (PF) magnitude, PF interval time T, and flow rise velocity V, to correctly identify the number of compressions. The compression number identification solution is shown in FIG. 2.

A peak pressure read for the first time is taken as a PF₁ value of the first time, and after entering the n^{th} compression (n>1), the system captures PF) and extracts a rise velocity Vₙ of the PFₙ. Only when the Vₙ is less than a threshold M₁, a possible RF is considered; otherwise, the PFₙ is discarded. Then, a time interval Tₙ is extracted to determine whether the Tₙ exceeds a threshold M₂. When the Tₙ is less than the M₂, an invalid PF is considered and is not recorded. It needs to be determined whether the PFₙ that meets conditions of V and T exceeds a threshold M₃, and when exceeding the M₃, a valid PF is determined, and a compression number is increased by one count. In the present example, the threshold M₁ is set to 1000 ml/s², the threshold M₂ is set to 0.3 s, and the threshold M₃ includes two parts: an average value of previous PFs in a case of less than five compressions, or an average value of previous five PFs in a case of more than five compressions. This solution can effectively eliminate the secondary wave after the RF, further accurately obtaining the number of compressions.

Certainly, in other examples, the values of the above thresholds can be set and adjusted based on specific circumstances.

In a specific control process, it is also necessary to establish a VCV mode for the portable ventilator suitable for CPR.

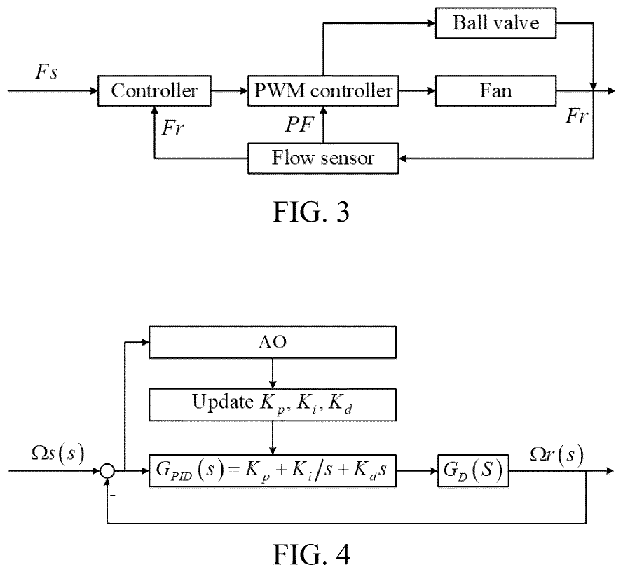

The VCV control processes of the portable emergency ventilator are shown in FIG. 3.

The flow sensor reads the flow rate of the ventilator, and feeds back the read actual flow rate Fr to the controller. During normal ventilation, the controller compares the actual flow rate Fr with the preset flow rate Fs to complete the real-time control of the PWM value required by the centrifugal fan, thereby adjusting the flow rate of the ventilator in real-time. During CPR, the PWM controller takes the PF value of the RF as input and re-adjusts the PWM value to complete the flow rate regulation during the compression period and relaxation period.

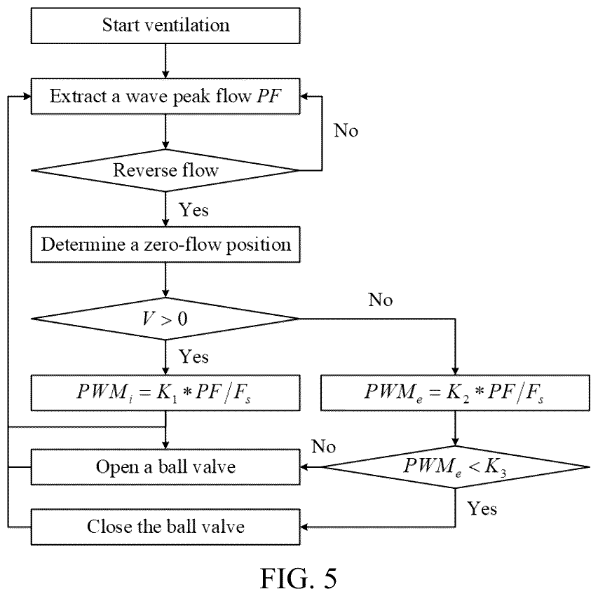

In the present example, the PWM control algorithm is divided into a normal ventilation algorithm under the “30:2” mode, as shown in FIG. 4, and a variable adjustment algorithm with the establishment of an advanced airway, as shown in FIG. 5.

The first PWM algorithm described in detail is the AO-PID, which is the PWM algorithm under the “30:2” mode.

The controller and the PWM controller jointly form the microcontroller 8.

In the present example, the fan 9 uses a micro direct-current brushless fan. In the “30:2” ventilation, the AO-PID control method is used to control the fan, so as to accurately control a preset reference flow rate. The AO-PID control algorithm adds the AO algorithm to the PID algorithm to optimize three parameters Kₚ, Kᵢ, and K_{d}, where Kₚ, Kᵢ, and K_{d} are proportional, integral, and differential coefficients, respectively. FIG. 4 shows a schematic flowchart of the AO-PID control algorithm that is specifically as follows:

The AO-PID algorithm randomly initializes a PID parameter matrix X, and the parameter matrix X is shown as (1).

$$X = [ \begin{matrix}{K}_{p 0} & {K}_{i 0} & {K}_{d 0} \\ ⋮ & ⋮ & ⋮ \\ {K}_{pm} & {K}_{im} & {K}_{dm}\end{matrix} ] \qquad ( 1 )$$

$${X}_{ij} = rand \times ( {UB}_{j} - {LB}_{j} ) + {LB}_{j} , i = 1 , 2 , … , m , j = 1 , 2 , 3 \qquad ( 2 )$$

The elements in the parameter matrix X are randomly generated according to formula (2), where UBⱼ and LBⱼ are upper and lower bounds of the parameters to be determined. In the present example, UBⱼ and LBⱼ are the upper and lower bounds of Kₚ, Kᵢ, and K_{d}. rand is a random number.

The first iteration is performed based on the following formulas:

$${X}_{1} ( t + 1 ) = {X}_{best} ( t ) ( 1 - t / T ) + ( {X}_{M} ( t ) - {X}_{best} ( t ) ) \times rand \qquad ( 3 )$$

$${X}_{M} ( t ) = \frac{1}{N} {\sum }_{i = 1}^{N} {X}_{i} ( t ) , M = 1 , 2 , … , dim \qquad ( 4 )$$

where X₁(t+1) is a solution for the next iteration of t, X_{best}(t) is a best solution obtained before the iteration, X_{M}(t) is the point average value of the current solutions at the t^{th} iteration, rand is a random number, and dim is a dimension of the problem, which is 3 in the present example. N is the total number of randomly generated problems m.

The second iteration is performed as follows according to formulas (5)-(12):

$${X}_{2} ( t + 1 ) = {X}_{best} ( t ) M ( D ) + {X}_{R} ( t ) + ( y - x ) \times rand \qquad ( 5 )$$

$$M ( D ) = s \times \frac{μ + σ}{{\vert v \vert}^{\frac{1}{β}}} \qquad ( 6 )$$

$$σ = \frac{Γ ( 1 + β ) \times sin ( \frac{πβ}{2} )}{Γ ( \frac{πβ}{2} ) \times β \times {2}^{( \frac{β - 1}{2} )}} \qquad ( 7 )$$

$$y = r \times cos ( θ ) \qquad ( 8 )$$

$$x = r \times sin ( θ ) \qquad ( 9 )$$

$$r = {r}_{1} + U + {D}_{1} 1 \qquad ( 10 )$$

$$θ = - ω \times {D}_{1} + {θ}_{1} \qquad ( 11 )$$

$${θ}_{1} = \frac{3 \times π}{2} \qquad ( 12 )$$

The iteration t generated in the second step is according to formula (5), where M(D) is an optimal value coefficient, D is a problem dimension, and X_{R}(t) is a random value from 1 to N. s is a fixed constant with a maximum range of 0.01. μ and v are random values between 0 and 1, and σ is a fixed constant with a maximum range of 1.5. x and y are used to describe the path shape in the search. r₁ is a value between 1 and 20, used to fix the number of search cycles. U is a variable multiplied by 0.00565, and D₁ is an integer from 1 to the maximum value of the search space variable (dim). ω is a small fixed value multiplied by a variable 0.005.

The third iteration is performed according to formula (13):

$${X}_{3} ( t + 1 ) = ( {X}_{best} ( t ) - {X}_{R} ( t ) ) \times α - rand + ( UB - LB ) \times rand + LB \times δ \qquad ( 13 )$$

Where, α and δ are two adjustment parameters, fixed between 0 and 1, and UB and LB represent the upper and lower bounds of a given problem.

The fourth iteration is performed according to formulas (14)-(17).

$${X}_{4} ( t + 1 ) = {Q}_{f} {X}_{best} ( t ) - {G}_{1} X ( t ) - ( {G}_{1} \times X ( t ) \times rand ) - {G}_{2} \times M ( D ) + rand \times {G}_{1} \qquad ( 14 )$$

$${Q}_{f} = t \frac{2 \times rand - 1}{{( 1 - T )}^{2}} \qquad ( 15 )$$

$${G}_{1} = 2 \times rand - 1 \qquad ( 16 )$$

$${G}_{2} = 2 \times ( 1 - \frac{t}{T} ) \qquad ( 17 )$$

The solution generated by the fourth iteration is X₄(t+1). The quality function used to balance the search strategy is Q_{f}, T is the total number of iterations, and t is the current number of iterations. G, decreases from 2 to 0, and rand ranges from 0 to 1.

After the flow rate of the ventilator during normal ventilation is controlled, to cope with the special flow rate and pressure changes in the CPR process of establishing an advanced airway mode, the ventilator needs to identify the relaxation and compression periods during compression based on the RF, the rotation speed of the fan is increased during the relaxation period, and the rotation speed is reduced or the fan is turned off during the compression period. The variable flow rate regulation solution is shown in FIG. 5. After ventilation begins, the ventilator extracts the peak flow PF value to determine whether there is a generation of the RF. Once the RF is confirmed, the zero position of flow rate of the RF is located, and then determining whether a flow acceleration V at the zero position is greater than 0. For the zero position of V being greater than 0, the ventilator determines the zero position as an inspiratory period, and for the zero position of V being less than 0, the ventilator determines the zero position as an expiratory period. For the inspiratory period, the ventilator adjusts the PWM value to PWMᵢ=K₁*PF/Fₛ; for the expiratory period, the ventilator adjusts the PWM value to PWMₑ=K₂*PF/Fₛ. Where, PWMᵢ and PWMₑ are PWM values of the fan at the inspiratory period and the expiratory period respectively, K₁ and K₂ are gain coefficients, PF is the value of the RF, and under normal ventilation conditions Fₛ is the target flow rate value. When PWMₑ of the expiratory period is less than the threshold K₃, the ventilator uses a ball valve to close the airway of the ventilator to block the ventilation, reducing the patient's airway pressure to avoid barotrauma.

As shown in FIG. 5, the occurrence of the reverse airflow RF is determined based on the peak flow rate PF; the zero position of flow rate is determined simultaneously. At the zero position of flow rate, the magnitude of the flow acceleration V is used to determine the PWM values during the compression period and the relaxation period. When the PWM value is too low; the ball valve is closed so as to close the airway of the ventilator, preventing the fan from stopping, thus speeding up the response of the ventilator.

Those skilled in the art should understand that the embodiments of the present invention may be provided as a method, a system, or a computer program product. Therefore, the present invention may use a form of hardware only embodiments, software only embodiments, or embodiments with a combination of software and hardware. Moreover, the present invention may be in a form of a computer program product that is implemented on one or more computer-usable storage media (including but not limited to a magnetic disk memory, a CD-ROM, an optical memory, and the like) that include computer-usable program code.

The present invention is described with reference to the flowcharts and/or block diagrams of the method, the device (system) and the computer program product according to the embodiments of the present invention. It should be understood that computer program instructions may be used to implement each process and/or each block in the flowcharts and/or the block diagrams and a combination of a process and/or a block in the flowcharts and/or the block diagrams. These computer program instructions may be provided for a general-purpose computer, a dedicated computer, an embedded processor, or a processor of any other programmable data processing device to generate a machine, such that the instructions executed by a computer or a processor of any other programmable data processing device enable generation of an apparatus for implementing a specific function in one or more processes in the flowcharts and/or in one or more blocks in the block diagrams.

These computer program instructions may be stored in a computer readable memory that can instruct the computer or any other programmable data processing device to work in a specific manner, such that the instructions stored in the computer readable memory generate an artifact that includes an instruction apparatus. The instruction apparatus implements a specific function in one or more processes in the flowcharts and/or in one or more blocks in the block diagrams.

These computer program instructions may be loaded onto a computer or another programmable data processing device, such that a series of operations and steps are performed on the computer or the another programmable device, thereby generating computer-implemented processing. Therefore, the instructions executed on the computer or another programmable device provides steps for implementing a specific function in one or more processes in the flowcharts and/or in one or more blocks in the block diagrams.

The above descriptions are merely preferred embodiments of the present invention and are not intended to limit the present invention. For those skilled in the art, various modifications and changes can be made to the present invention. Any modification, equivalent replacement, improvement, or the like made within the spirit and principle of the present invention shall fall within the protection scope of the present invention.

Although the specific implementations of the present invention have been described with reference to the accompanying drawings, they do not limit the scope of the present invention. Those skilled in the art should understand that various modifications or alterations made based on the technical solutions of the present invention, without creative efforts, still fall within the scope of protection of the present invention.

1. A portable ventilator suitable for cardiopulmonary resuscitation,
comprising a fan, an air-oxygen mixing device, a pipeline, a controller, and a flow sensor, wherein:
an air-inlet of the fan is connected to the air-oxygen mixing device, and an outlet is connected to a first end of an oxygen sensor;
a second end of the oxygen sensor is connected to an air-inlet of the flow sensor being used to monitor a flow rate, an outlet of the flow sensor is connected to the pipeline, and a control switch is provided on pipeline and is used to close the pipeline;
a pressure sensor is further provided on the pipeline and is used to monitor an air pressure in the pipeline; and
the controller is communicated with the flow sensor and the pressure sensor and is configured to perform a feedback control on the fan based on monitoring data of sensors, ensuring provision of a constant flow rate of air.

2. The portable ventilator suitable for cardiopulmonary resuscitation according to claim 1, wherein a first end of the air-oxygen mixing device is connected to the atmosphere and a second end is connected to a proportional solenoid valve, and an inlet of the proportional solenoid valve is connected to an oxygen source.

3. The portable ventilator suitable for cardiopulmonary resuscitation according to claim 1, wherein the controller is configured to compare an actual flow rate obtained by the flow sensor with a preset flow rate, and adjust a flow rate of the ventilator based on a difference of the comparison, and generate a PWM (Pulse-Width Modulation) value required by the fan.

4. The portable ventilator suitable for cardiopulmonary resuscitation according to claim 1, wherein the controller is configured to determine whether there is a reverse airflow caused by the compression based on time and flow rate parameters during a cardiopulmonary resuscitation (CPR), count reverse airflows caused by compressions, read a number of the compressions when no advanced airway is established, and carry out ventilations after the compressions.

5. The portable ventilator suitable for cardiopulmonary resuscitation according to claim 1, wherein the controller is configured to determine optimized control parameters after the advanced airway is established, control the flow rate in the airway, determine a compression period and a relaxation period of an External Chest Compression (ECC) based on the reverse airflow caused by the compression, and reduce a rotation speed of the fan or turn off the fan during the compression period, or increase the rotation speed of the fan during the relaxation period.

6. A control method for portable ventilator according to claim 1, comprising the following steps:
obtaining monitoring data of a flow sensor and a pressure sensor;
comparing an actual flow rate obtained by the flow sensor with a preset flow rate, and adjusting a flow rate of a ventilator based on a comparison difference to generate a PWM value required by a fan;
during a cardiopulmonary resuscitation (CPR), determining whether there is a reverse airflow caused by the compression based on time and flow rate parameters, counting reverse airflows caused by compressions; when no advanced airway is established, reading a number of the compressions, and carrying out ventilations after the compressions; and
determining optimized control parameters after the advanced airway is established, controlling the flow rate in the airway; determining a compression period and a relaxation period of an External Chest Compression (ECC) based on the reverse airflow caused by the compression, and reducing a rotation speed of the fan or turn off the fan during the compression period, or increasing the rotation speed of the fan during the relaxation period.

7. The control method according to claim 6, wherein determining whether there is the reverse airflow caused by the compression based on time and flow rate parameters specifically comprises:
taking a peak pressure read for the first time as a peak flow value of the first time, after entering the n^{th} compression, obtaining a peak flow value of the n^{th} time, and extracting a rise velocity of the peak flow value of the n^{th} time, wherein n is greater than 1;
when the rise velocity is less than a first threshold, considering that there may be the reverse airflow caused by the compression, otherwise discarding the peak flow value of the n^{th} time;
extracting a time interval Tₙ, determining whether the Tₙ exceeds a second threshold; when the Tₙ is less than the second threshold, determining as an invalid peak flow, and not recording; and
determining whether the peak flow value of the n^{th} time that meets conditions of the rise velocity and the time interval exceeds a third threshold, determining the peak flow value of the n^{th} time that exceeds the third threshold as a valid peak flow, and increasing the compressions by one count.

8. The control method according to claim 6, wherein determining optimized control parameters specifically comprises iteratively calculating optimized proportional, integral, and differential coefficients by using an AO-PID (Analog Output-Proportional Integral Derivative) control algorithm.

9. The control method according to claim 6, wherein re-adjusting the PWM value based on the peak flow value of the reverse airflow caused by the compression, to achieve a flow rate adjustment in the compression period and the relaxation period.

10. The control method according to claim 6, wherein extracting the peak flow value to determine whether there is a generation of the reverse airflow caused by the compression, locating a zero position of flow rate of the reverse airflow caused by the compression after the reverse airflow caused by the compression has been determined, simultaneously determining whether a flow acceleration at the zero position is greater than 0, wherein if the flow acceleration at the zero position is greater than 0, determining, by the ventilator, that the zero position is an inspiratory period, and if the flow acceleration of the zero position is less than 0, determining that the zero position is an expiratory period; wherein
for the inspiratory period, adjusting the PWM value to PWMᵢ=K₁*PF/Fₛ; and for the expiratory period, adjusting the PWM value to PWMₑ=K₂*PF/Fₛ; where, PWMᵢ and PWMₑ are PWM values of the fan at the inspiratory period and the expiratory period respectively, K₁ and K₂ are gain coefficients, PF is the peak flow value of the reverse airflow caused by the compression; under normal ventilation conditions, Fₛ is a target flow rate value; and, when the PWMₑ of the expiratory period is less than a set threshold, closing the pipeline.

11. The portable ventilator suitable for cardiopulmonary resuscitation according to claim 3, wherein the controller is configured to determine whether there is a reverse airflow caused by the compression based on time and flow rate parameters during a cardiopulmonary resuscitation (CPR), count reverse airflows caused by compressions, read a number of the compressions when no advanced airway is established, and carry out ventilations after the compressions.

12. The portable ventilator suitable for cardiopulmonary resuscitation according to claim 3, wherein the controller is configured to determine optimized control parameters after the advanced airway is established, control the flow rate in the airway, determine a compression period and a relaxation period of an External Chest Compression (ECC) based on the reverse airflow caused by the compression, and reduce a rotation speed of the fan or turn off the fan during the compression period, or increase the rotation speed of the fan during the relaxation period.

13. The control method according to claim 9, wherein extracting the peak flow value to determine whether there is a generation of the reverse airflow caused by the compression, locating a zero position of flow rate of the reverse airflow caused by the compression after the reverse airflow caused by the compression has been determined, simultaneously determining whether a flow acceleration at the zero position is greater than 0, wherein if the flow acceleration at the zero position is greater than 0, determining, by the ventilator, that the zero position is an inspiratory period, and if the flow acceleration of the zero position is less than 0, determining that the zero position is an expiratory period; wherein
for the inspiratory period, adjusting the PWM value to PWMᵢ=K₁*PF/Fₛ; and for the expiratory period, adjusting the PWM value to PWMₑ=K₂*PF/Fₛ; where, PWMᵢ and PWMₑ are PWM values of the fan at the inspiratory period and the expiratory period respectively, K₁ and K₂ are gain coefficients, PF is the peak flow value of the reverse airflow caused by the compression; under normal ventilation conditions, Fₛ is a target flow rate value; and, when the PWMₑ of the expiratory period is less than a set threshold, closing the pipeline.
